(19) United States
(12) Patent Application Publication
(10) Pub. No.: US 2026/0200826 A1
(43) Pub. Date: Jul. 16, 2026

(54) METHOD FOR SYNTHESIZING FLUOROALKYL-SUBSTITUTED 4,4'-DIAMINODIPHENYLMETHANE COMPOUND

(71) Applicant: Hainan University, Haikou City (CN)

(72) Inventors: Jinshan LI, Haikou City (CN); Chunman JIA, Haikou City (CN); Ziyan WU, Haikou City (CN); Xindi LI, Haikou City (CN)

(21) Appl. No.: 19/449,023

(22) Filed: Jan. 14, 2026

(30) Foreign Application Priority Data
Jan. 15, 2025 (CN) 202510063629.9

(51) Int. Cl. C07C 209/74; C07D 311/82

(52) U.S. Cl. CPC C07C 209/74; C07D 311/82; C07C 2601/16

(57) ABSTRACT

The present invention provides a method for synthesizing a fluoroalkyl-substituted 4,4′-diaminodiphenylmethane compound, which includes: under the action of a catalyst, allowing an amine compound to react with a fluorine-containing aldehyde compound in an organic solvent, and after completion of the reaction, performing post-treatment to obtain the fluoroalkyl-substituted 4,4′-diaminodiphenylmethane compound. The present invention has mild reaction conditions, simple operations, high reaction efficiency, inexpensive and readily available raw materials, good atom economy, and no generation of waste, thus having a large-scale application prospect. Furthermore, a fluoroalcohol solvent is recycled through a simple distillation operation, which effectively reduces the cost of a large amount of the solvent, improves process safety, and is conducive to efficient and green industrial production.

### FIELD OF TECHNOLOGY

The present invention belongs to the field of organic synthesis, and particularly relates to a method for synthesizing a fluoroalkyl-substituted 4,4′-diaminodiphenylmethane compound.

### BACKGROUND

Polyimide is a high-performance engineering material, and due to a rigid imide structure, has good high-temperature resistance, chemical stability, mechanical properties, electrical properties, radiation resistance, and self-lubricating property, which has been widely used in industries such as aerospace, machinery, nanotechnology, separation membranes, electrical engineering, and microelectronics. However, the polyimide is high in melting point, poor in solubility, difficult to mold and process, and high in production cost, and due to a highly conjugated aromatic structure and an intermolecular charge transfer complex, has strong coloration, reduced optical transparency, and a high dielectric constant, thus limiting its application range in technical fields such as flexible electronic products and flexible printed circuit boards.

Introducing a fluorine-containing substituent into a molecular structure of the polyimide can effectively overcome these shortcomings, reduce optical loss, the dielectric constant, and a moisture absorption rate, and meanwhile, improve the solubility, transparency, and thermal stability, thereby enabling unique advantages and broad development prospects in fields such as optoelectronics and aerospace. Trifluoromethyl-substituted 4,4′-diaminodiphenylmethane, as an important fluorine-containing polyimide monomer, is disclosed in Patent Application No. CN113396137A, entitled “MANUFACTURING METHOD FOR 1,1,1-TRIFLUORO-2,2-DIARYLETHANE AND 1,1,1-TRIFLUORO-2,2-DIARYLETHANE”, which uses aniline and trifluoroacetaldehyde as raw materials. Under anhydrous conditions, an anhydrous HF gas is introduced to serve as a catalyst, and a large amount of trifluoromethanesulfonic acid is added. Meanwhile, special equipment such as a stainless steel high-pressure reactor is required for a reaction under high temperature and high pressure, which brings great inconvenience to production and operation, resulting in dangerous and cumbersome operation. After completion of the reaction, neutralization with a 48% potassium hydroxide aqueous solution is required, which generates a large amount of wastewater. Furthermore, the catalyst used is the HF gas, which has high corrosion and high risk and is difficult to recycle, thus causing severe environmental pollution.

In summary, current methods for synthesizing the trifluoromethyl-substituted 4,4′-diaminodiphenylmethane have some problems, and it is particularly important to develop a synthesis method for synthesizing trifluoromethyl-substituted and other fluoroalkyl-substituted 4,4′-diaminodiphenylmethane that has simple operations, high safety, a low cost, and environmental friendliness. Furthermore, there are no reports on the synthesis of fluoroalkyl, such as difluoromethyl and pentafluoroethyl, substituted 4,4′-diaminodiphenylmethane compounds.

### SUMMARY

The present invention provides a method for synthesizing a fluoroalkyl-substituted 4,4′-diaminodiphenylmethane compound. The method has readily available raw materials, simple operation, mild reaction conditions, high safety, a high reaction yield, and good atom economy, thus having a large-scale application prospect.

To achieve the above objective, technical solutions adopted in the present invention are as follows.

A method for synthesizing a fluoroalkyl-substituted 4,4′-diaminodiphenylmethane compound includes:
under the action of a catalyst, allowing an amine compound to react with a fluorine-containing aldehyde compound in an organic solvent, and after completion of the reaction, performing post-treatment to obtain the fluoroalkyl-substituted 4,4′-diaminodiphenylmethane compound having a structure of general formula [1]:

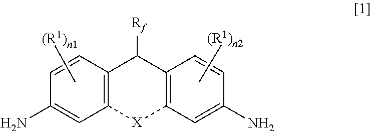

where in the general formula [1], n₁ and n₂ are respectively and independently an integer of 0-4, and in cases where multiple R₁ are present, respectively and independently represent a substituent;
R¹ is independently selected from hydrogen, alkyl, alkoxy, cycloalkyl, aryl, propenyl, halogen, hydroxyl, benzyl, thioalkyl, and an ester group;
R_{f} is independently selected from difluoromethyl, trifluoromethyl, trifluoroethyl, tetrafluoroethyl, pentafluoroethyl, heptafluoropropyl, and nonafluorobutyl; and
X is independently selected from methylene, oxygen, or sulfur.

As a preference, the amine compound includes a mono-substituted, multi-substituted, or unsubstituted aromatic amine compound and a diphenylamine compound.

As a further preference, in the mono-substituted, multi-substituted, or unsubstituted aromatic amine compound, each substituent is independently selected from hydrogen, methyl, ethyl, tert-butyl, isopropyl, alkoxy, cycloalkyl, phenyl, propenyl, halogen, hydroxyl, benzyl, thioalkyl, an ester group, and trifluoromethoxy; and the diphenylamine compound is diaminodiphenylmethane, oxydiphenylamine, or thiodiphenylamine.

As a preference, the fluorine-containing aldehyde compound includes difluoroacetaldehyde hydrate, difluoroacetaldehyde ethyl hemiacetal, trifluoroacetaldehyde hydrate, trifluoroacetaldehyde methyl hemiacetal, trifluoroacetaldehyde ethyl hemiacetal, 3,3,3-trifluoropropionaldehyde, 2,2,3,3-tetrafluoropropionaldehyde hydrate, pentafluoropropionaldehyde hydrate, heptafluorobutyraldehyde hydrate, or nonafluorovaleraldehyde hydrate.

As a preference, the catalyst is a Lewis acid or Brønsted acid.

As a further preference, the Lewis acid is selected from phenylboronic acid, boric acid, trimethyl borate, triethyl borate, triisopropyl borate, tributyl borate, triphenyl borate, boron trifluoride diethyl etherate, tris(2,4-bis(trifluoromethyl)phenyl)borane, tris(pentafluorophenyl)borane, tris(2,2,2-trifluoroethyl)borate, triphenylborane, tris(hexafluoroisopropyl)borate, tris(2,3,5,6-tetrafluoro-4-(trifluoromethyl)phenyl)borane, tris(2,4-bis(trifluoromethyl)phenyl)borane, trimethylsilyl trifluoromethanesulfonate, tert-butyldimethylsilyl trifluoromethanesulfonate, trimethylsilyl acetate, trimethylsilylmethanesulfonate, N-(trimethylsilyl)bis(trifluoromethanesulfonyl)imide, (pentafluorophenyl)bis(trifluoromethanesulfonyl)trimethylsilylmethane, di-tert-butyl isobutylsilyl trifluoromethanesulfonate, tert-butyldiphenylsilyl trifluoromethanesulfonate, tert-butyldimethylsilyl trifluoromethanesulfonate, 2-(trimethylsilyl)phenyl trifluoromethanesulfonate, triisopropylsilyl trifluoromethanesulfonate, diisopropylsilyl bis(trifluoromethanesulfonate), diethylisopropylsilyl trifluoromethanesulfonate, di-tert-butylsilyl bis(trifluoromethanesulfonate), triethylsilyl trifluoromethanesulfonate, tris(2,6-difluorophenyl)borane, tris(2,5-bis(trifluoromethyl)phenyl)borane, 2,4,6-tris(3-(trifluoromethyl)phenyl)-1,3,5,2,4,6-trioxatriborinane, triphenylmethyl tetrakis(pentafluorophenyl)borate, bis(perfluorophenyl)borane, tris(2,4,6-trifluorophenyl)borane, tris[3,5-bis(trifluoromethyl)phenyl]borane, tris(2,2′,2″-perfluorobiphenyl)borane, bis(pentafluorophenyl)-(2-perfluorobiphenyl)borane, or tris(2-perfluoronaphthyl)borane; and
the Brønsted acid is selected from acetic acid, benzoic acid, benzenesulfonic acid, p-toluenesulfonic acid, methanesulfonic acid, camphorsulfonic acid, Amberlyst 15 ion exchange resin, diphenyl phosphate, binaphthol phosphate, trifluoroacetic acid, trifluoromethanesulfonic acid, lactic acid, oxalic acid, sulfuric acid, hydrochloric acid, perchloric acid, phosphoric acid, or polyphosphoric acid.

As a preference, the organic solvent is 2,2-difluoroethanol, hexafluoroisopropanol, hexafluoro-2-methylisopropanol, hexafluoro-2-phenylisopropanol, trifluoroethanol, perfluoroalkylethanol, perfluoro-tert-butanol, ethylene glycol, tetrafluoropropanol, tetrafluorobutanediol, hexafluorobutanol, or octafluoropentanol.

As a preference, a reaction temperature is from room temperature to 130° C., and a reaction time is 12-24 h. More preferably, the reaction is at 45-90° C. for 12-20 h.

As a preference, a molar ratio of the amine compound to the fluorine-containing aldehyde compound is (1-2.4):1.

As a preference, a molar ratio of the fluorine-containing aldehyde compound to the catalyst is 1:(0-0.2).

As a preference, the post-treatment includes: after completion of the reaction, removing the solvent under reduced pressure, and recrystallizing a crude reaction product to obtain the fluoroalkyl-substituted 4,4′-diaminodiphenylmethane compound.

As a further preference, a solvent used for the recrystallizing includes ethanol, methanol, toluene, ethyl acetate, and chloroform.

As a further preference, after completion of the reaction, a fluoroalcohol solvent is recovered through a simple distillation operation.

Compared with the prior art, the present invention has the following beneficial effects.

The method for synthesizing a fluoroalkyl-substituted 4,4′-diaminodiphenylmethane compound provided by the present invention effectively solves the problems that traditional methods have the requirement of reactions under anhydrous conditions, the use of hydrogen fluoride as a catalyst, high temperature and high pressure, high operation risk, and complex post-treatment, etc. In the present invention, by using the Lewis acid or Brønsted acid as the catalyst, the reaction does not require anhydrous conditions, and the method has mild reaction conditions, simple operations, high reaction efficiency, inexpensive and readily available raw materials, good atom economy, and no generation of waste, thus having a large-scale application prospect. Furthermore, the fluoroalcohol solvent is recycled through a simple distillation operation, which effectively reduces the cost of a large amount of the solvent, improves process safety, and is conducive to efficient and green industrial production.

### DESCRIPTION OF THE EMBODIMENTS

The present invention is described in detail below in conjunction with specific embodiments. The following specific examples are provided to further facilitate understanding of the present invention by those skilled in the art, but do not in any way limit the present invention.

### Example 1

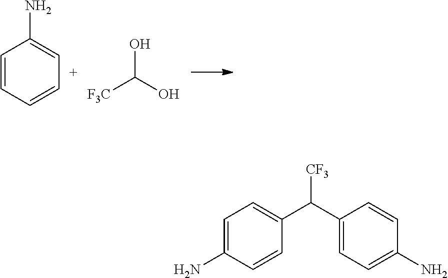 [SMILES: Nc1ccccc1 ; OC(O)C(F)(F)F ; Nc1ccc(C(c2ccc(N)cc2)C(F)(F)F)cc1]

Aniline (0.76 mol, 2 equiv), a trifluoroacetaldehyde hydrate (44.1 g, 0.38 mol), trifluoroethanol (640 mL), and phenylboronic acid (0.038 mol) were sequentially added into a 1,000 mL reaction flask to carry out a reaction at 45° C. According to monitoring by thin layer chromatography (TLC) plate spotting, the reaction was completed at 12 h. After completion of the reaction, the solvent was recovered by rotary evaporation, and a crude reaction product was recrystallized with toluene to obtain 80.8 g of a product (4,4′-(2,2,2-trifluoroethane-1,1-diyl)dianiline) of the above formula with a yield of 80%.

¹H NMR (400 MHz, CDCl₃) δ 7.13 (d, J=8.2 Hz, 4H), 6.64 (d, J=8.4 Hz, 4H), 4.46 (q, J=10.1 Hz, 1H), 3.65 (s, 4H); ¹⁹F NMR (376 MHz, CDCl₃) δ −66.41 (d, J=9.9 Hz, 3F); ¹³C NMR (100 MHz, CDCl₃) δ 145.8, 129.9, 126.6 (d, ¹J_{C-F}=281.8 Hz), 125.8, 115.1, 53.9 (q, ²J_{C-F}=27.3 Hz); HRMS (ESI) m/z: [M+H]⁺ calculated value C₁₄H₁₄F₃N₂ 267.1104; measured value: 267.1108.

### Example 2

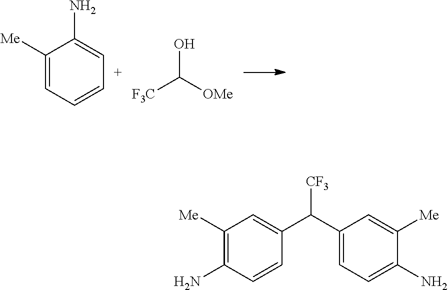 [SMILES: Cc1ccccc1N ; COC(O)C(F)(F)F ; Cc1cc(C(c2ccc(N)c(C)c2)C(F)(F)F)ccc1N]

2-methylaniline (0.76 mol, 2 equiv), trifluoroacetaldehyde methyl hemiacetal (49.4 g, 0.38 mol), trifluoroethanol (640 mL), and tris[3,5-bis(trifluoromethyl)phenyl]borane (0.038 mol) were sequentially added into a 1,000 mL reaction flask to carry out a reaction at 80° C. According to monitoring by TLC plate spotting, the reaction was completed at 12 h. After completion of the reaction, the solvent was recovered by rotary evaporation, and a crude reaction product was recrystallized with ethanol to obtain 107.2 g of a product (4,4′-(2,2,2-trifluoroethane-1,1-diyl)bis(2-methylaniline)) of the above formula with a yield of 96%.

¹H NMR (400 MHz, CDCl₃) δ 7.04 (d, J=8.3 Hz, 4H), 6.63 (d, J=7.8 Hz, 2H), 4.42 (q, J=10.3 Hz, 1H), 3.58 (s, 4H), 2.14 (s, 6H); ¹⁹F NMR (376 MHz, CDCl₃) δ −66.24 (d, J=10.2 Hz, 3F); ¹³C NMR (100 MHz, CDCl₃) δ 144.0, 131.1, 127.4, 126.6 (d, ¹J_{C-F}=281.5 Hz), 126.0, 122.3, 114.9, 54.1 (q, ²J_{C-F}=27.2 Hz), 17.4; HRMS (ESI) m/z: [M+H]⁺ calculated value C₁₆H₁₈F₃N₂ 295.1417; measured value: 295.1413.

### Example 3

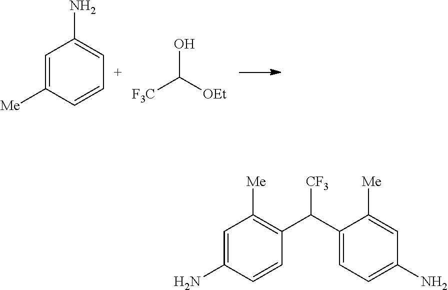 [SMILES: Cc1cccc(N)c1 ; CCOC(O)C(F)(F)F ; Cc1cc(N)ccc1C(c1ccc(N)cc1C)C(F)(F)F]

3-methylaniline (0.76 mol, 2 equiv), trifluoroacetaldehyde ethyl hemiacetal (54.7 g, 0.38 mol), hexafluoroisopropanol (640 mL), and tris(2,4-bis(trifluoromethyl)phenyl)borane (0.019 mol) were sequentially added into a 1,000 mL reaction flask to carry out a reaction at 65° C. According to monitoring by TLC plate spotting, the reaction was completed at 14 h. After completion of the reaction, the solvent was removed by rotary evaporation, and a crude reaction product was recrystallized with ethyl acetate to obtain 72.6 g of a product (4,4′-(2,2,2-trifluoroethane-1,1-diyl)bis(3-methylaniline)) of the above formula with a yield of 65%.

¹H NMR (400 MHz, CDCl₃) δ 7.17 (d, J=8.3 Hz, 2H), 6.53-6.46 (m, 4H), 4.82 (q, J=9.7 Hz, 1H), 3.59 (s, 4H), 2.21 (s, 6H); ¹⁹F NMR (376 MHz, CDCl₃) δ −65.10 (d, J=9.8 Hz, 3F); ¹³C NMR (100 MHz, CDCl₃) δ 145.5, 137.8, 129.6, 127.2 (d, ¹J_{C-F}=282.2 Hz), 124.3, 117.2, 112.8, 45.9 (q, ²J_{C-F}=26.8 Hz), 19.7; HRMS (ESI) m/z: [M+H]⁺ calculated value C₁₆H₁₈F₃N₂ 295.1417; measured value: 295.1416.

### Example 4

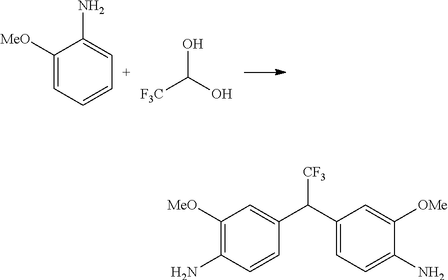 [SMILES: COc1ccccc1N ; OC(O)C(F)(F)F ; COc1cc(C(c2ccc(N)c(OC)c2)C(F)(F)F)ccc1N]

2-methoxyaniline (0.84 mol, 2.2 equiv), a trifluoroacetaldehyde hydrate (44.1 g, 0.38 mol), perfluoro-tert-butanol (640 mL), and trimethyl borate (0.038 mol) were sequentially added into a 1,000 mL reaction flask to carry out a reaction at 50° C. According to monitoring by TLC plate spotting, the reaction was completed at 12 h. After completion of the reaction, the solvent was removed by rotary evaporation, and a crude reaction product was recrystallized with methanol to obtain 107.8 g of a product of the above formula with a yield of 87%.

### Example 5

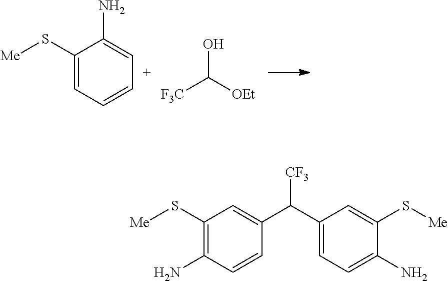 [SMILES: CSc1ccccc1N ; CCOC(O)C(F)(F)F ; CSc1cc(C(c2ccc(N)c(SC)c2)C(F)(F)F)ccc1N]

2-methylthioaniline (0.76 mol, 2 equiv), trifluoroacetaldehyde ethyl hemiacetal (54.7 g, 0.38 mol), hexafluoroisopropanol (640 mL), and triethyl borate (0.038 mol) were sequentially added into a 1,000 mL reaction flask to carry out a reaction at 90° C. According to monitoring by TLC plate spotting, the reaction was completed at 12 h. After completion of the reaction, the solvent was recovered by rotary evaporation, and a crude reaction product was recrystallized with ethanol to obtain 83.3 g of a product of the above formula with a yield of 61%.

### Example 6

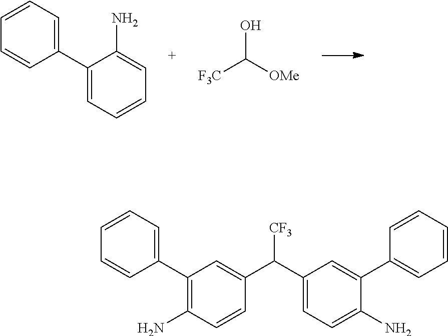 [SMILES: Nc1ccccc1-c1ccccc1 ; COC(O)C(F)(F)F ; Nc1ccc(C(c2ccc(N)c(-c3ccccc3)c2)C(F)(F)F)cc1-c1ccccc1]

2-aminobiphenyl (0.76 mol, 2 equiv), trifluoroacetaldehyde methyl hemiacetal (49.4 g, 0.38 mol), 2,2-difluoroethanol (640 mL), and tris(2,2,2-trifluoroethyl)borate (0.038 mol) were sequentially added into a 1,000 mL reaction flask to carry out a reaction at 80° C. According to monitoring by TLC plate spotting, the reaction was completed at 15 h. After completion of the reaction, the solvent was removed by rotary evaporation, and a crude reaction product was recrystallized with chloroform to obtain 150.9 g of a product of the above formula with a yield of 95%.

### Example 7

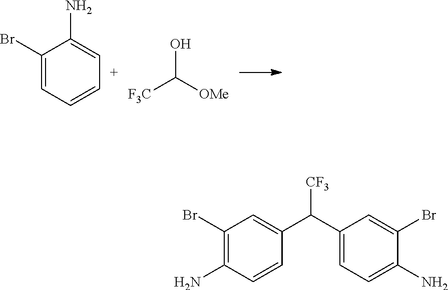 [SMILES: Nc1ccccc1Br ; COC(O)C(F)(F)F ; Nc1ccc(C(c2ccc(N)c(Br)c2)C(F)(F)F)cc1Br]

2-bromoaniline (0.76 mol, 2 equiv), trifluoroacetaldehyde methyl hemiacetal (49.4 g, 0.38 mol), hexafluoro-2-methylisopropanol (640 mL), and tris(hexafluoroisopropyl)borate (0.076 mol) were sequentially added into a 1,000 mL reaction flask to carry out a reaction at 65° C. According to monitoring by TLC plate spotting, the reaction was completed at 12 h. After completion of the reaction, the solvent was recovered by rotary evaporation, and a crude reaction product was recrystallized with ethanol to obtain 82.5 g of a product of the above formula with a yield of 51%.

### Example 8

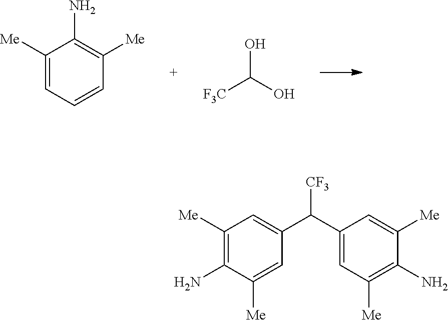 [SMILES: Cc1cccc(C)c1N ; OC(O)C(F)(F)F ; Cc1cc(C(c2cc(C)c(N)c(C)c2)C(F)(F)F)cc(C)c1N]

3,5-dimethylaniline (0.76 mol, 2 equiv), a trifluoroacetaldehyde hydrate (44.1 g, 0.38 mol), hexafluoroisopropanol (640 mL), and boron trifluoride diethyl etherate (0.038 mol) were sequentially added into a 1,000 mL reaction flask to carry out a reaction at 50° C. According to monitoring by TLC plate spotting, the reaction was completed at 15 h. After completion of the reaction, the solvent was recovered by rotary evaporation, and a crude reaction product was recrystallized with ethanol to obtain 118.7 g of a product (4,4′-(2,2,2-trifluoroethane-1,1-diyl)bis(2,6-dimethylaniline)) of the above formula with a yield of 97%.

¹H NMR (400 MHz, CDCl₃) δ 6.95 (s, 4H), 4.38 (q, J=10.4 Hz, 1H), 3.59 (s, 4H), 2.17 (s, 12H); ¹⁹F NMR (376 MHz, CDCl₃) δ −66.09 (d, J=10.3 Hz, 3F); ¹³C NMR (100 MHz, CDCl₃) δ 142.1, 128.8, 126.7 (d, ¹J_{C-F}=281.1 Hz), 125.4, 121.7, 54.2 (q, ²J_{C-F}=27.0 Hz), 17.7; HRMS (ESI) m/z: [M+H]⁺ calculated value C₁₈H₂₂F₃N₂ 323.1730; measured value: 323.1731.

### Example 9

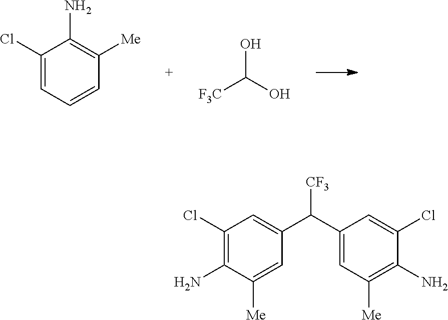 [SMILES: Cc1cccc(Cl)c1N ; OC(O)C(F)(F)F ; Cc1cc(C(c2cc(C)c(N)c(Cl)c2)C(F)(F)F)cc(Cl)c1N]

2-chloro-6-methylaniline (0.76 mol, 2 equiv), a trifluoroacetaldehyde hydrate (44.1 g, 0.38 mol), perfluoroalkylethanol (640 mL), and triisopropylsilyl trifluoromethanesulfonate (0.038 mol) were sequentially added into a 1,000 mL reaction flask to carry out a reaction at 65° C. According to monitoring by TLC plate spotting, the reaction was completed at 18 h. After completion of the reaction, the solvent was removed by rotary evaporation, and a crude reaction product was recrystallized with toluene to obtain 132.1 g of a product (4,4′-(2,2,2-trifluoroethane-1,1-diyl)bis(2-chloro-6-methylaniline)) of the above formula with a yield of 96%.

¹H NMR (400 MHz, CDCl₃) δ 7.12 (s, 2H), 6.92 (s, 2H), 4.35 (q, J=9.9 Hz, 1H), 4.00 (s, 4H), 2.18 (s, 6H); ¹⁹F NMR (376 MHz, CDCl₃) δ −66.29 (d, J=9.8 Hz, 3F); ¹³C NMR (100 MHz, CDCl₃) δ 140.8, 129.3, 127.3, 126.1 (d, ¹J_{C-F}=281.8 Hz), 125.3, 123.6, 119.0, 53.5 (q, ²J_{C-F}=27.6 Hz), 18.1; HRMS (ESI) m/z: [M+H]⁺ calculated value C₁₆H₁₆F₃N₂Cl₂ 363.0637; measured value: 363.0640.

### Example 10

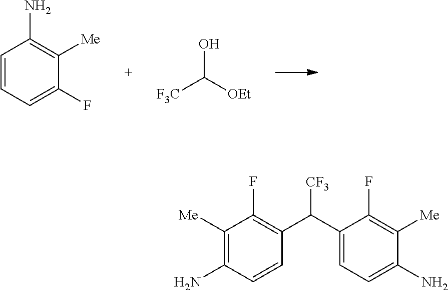 [SMILES: Cc1c(N)cccc1F ; CCOC(O)C(F)(F)F ; Cc1c(N)ccc(C(c2ccc(N)c(C)c2F)C(F)(F)F)c1F]

2-methyl-3-fluoroaniline (0.76 mol, 2 equiv), trifluoroacetaldehyde ethyl hemiacetal (54.7 g, 0.38 mol), hexafluoroisopropanol (640 mL), and bis(perfluorophenyl)borane (0.019 mol) were sequentially added into a 1,000 mL reaction flask to carry out a reaction at 45° C. According to monitoring by TLC plate spotting, the reaction was completed at 12 h. After completion of the reaction, the solvent was recovered by rotary evaporation, and a crude reaction product was recrystallized with ethanol to obtain 95.3 g of a product of the above formula with a yield of 76%.

### Example 11

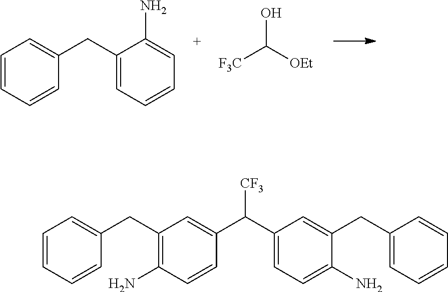 [SMILES: Nc1ccccc1Cc1ccccc1 ; CCOC(O)C(F)(F)F ; Nc1ccc(C(c2ccc(N)c(Cc3ccccc3)c2)C(F)(F)F)cc1Cc1ccccc1]

O-benzylaniline (0.8 mol, 2.1 equiv), trifluoroacetaldehyde ethyl hemiacetal (54.7 g, 0.38 mol), tetrafluoropropanol (640 mL), and tert-butyldimethylsilyl trifluoromethanesulfonate (0.038 mol) were sequentially added into a 1,000 mL reaction flask to carry out a reaction at 65° C. According to monitoring by TLC plate spotting, the reaction was completed at 18 h. After completion of the reaction, the solvent was recovered by rotary evaporation, and a crude reaction product was recrystallized with ethanol to obtain 155.9 g of a product (4,4′-(2,2,2-trifluoroethane-1,1-diyl)bis(2-benzylaniline)) of the above formula with a yield of 92%.

¹H NMR (400 MHz, CDCl₃) δ 7.30 (t, J=7.2 Hz, 4H), 7.26-7.15 (m, 6H), 7.14-7.07 (m, 4H), 6.64 (d, J=8.1 Hz, 2H), 4.49 (q, J=10.2 Hz, 1H), 3.90 (s, 4H), 3.37 (s, 4H); ¹⁹F NMR (376 MHz, CDCl₃) δ −66.24 (d, J=10.1 Hz, 3F); ¹³C NMR (100 MHz, CDCl₃) δ 144.2, 139.0, 131.7, 128.7, 128.4, 128.2, 126.4, 126.6 (d, ¹J_{C-F}=281.9 Hz), 126.0, 124.9, 115.9, 54.1 (q, ²J_{C-F}=27.2 Hz), 38.2; HRMS (ESI) m/z: [M+H]⁺ calculated value C₂₈H₂₆F₃N₂ 447.2043; measured value: 447.2050.

### Example 12

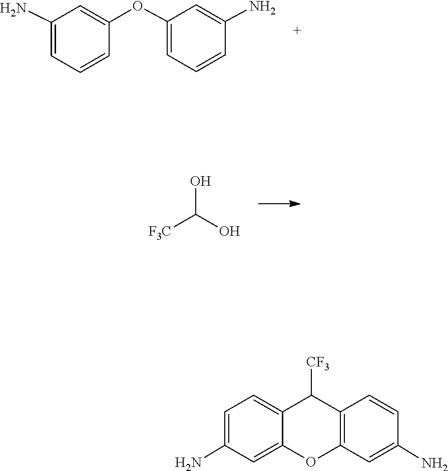 [SMILES: Nc1cccc(Oc2cccc(N)c2)c1 ; OC(O)C(F)(F)F ; Nc1ccc2c(c1)Oc1cc(N)ccc1C2C(F)(F)F]

3,3′-oxydiphenylamine (0.38 mol, 1 equiv), a trifluoroacetaldehyde hydrate (44.1 g, 0.38 mol), tetrafluoropropanol (640 mL), and boric acid (0.038 mol) were sequentially added into a 1,000 mL reaction flask to carry out a reaction at 65° C. According to monitoring by TLC plate spotting, the reaction was completed at 18 h. After completion of the reaction, the solvent was recovered by rotary evaporation, and a crude reaction product was recrystallized with ethanol to obtain 54.2 g of a product (9-(trifluoromethyl)-9H-xanthene-3,6-diamine) of the above formula with a yield of 51%.

¹H NMR (400 MHz, CDCl₃) δ 7.30 (d, J=8.3 Hz, 1H), 7.11 (t, J=8.1 Hz, 1H), 6.47-6.40 (m, 2H), 6.32 (s, 1H), 6.13 (d, J=1.9 Hz, 1H), 5.28 (q, J=7.0 Hz, 1H), 3.73 (s, 4H). ¹⁹F NMR (376 MHz, CDCl₃) δ −78.02 (d, J=6.8 Hz, 3F). ¹³C NMR (100 MHz, CDCl₃) δ 156.7, 148.0, 130.5, 129.8 (d, ¹J_{C-F}=281.5 Hz), 110.0, 105.1, 67.9 (q, ²J_{C-F}=27.2 Hz), 29.7. HRMS (ESI) m/z: [M+H]⁺ calculated value C₁₄H₁₂F₃N₂O 281.0896; measured value: 281.0899.

### Example 13

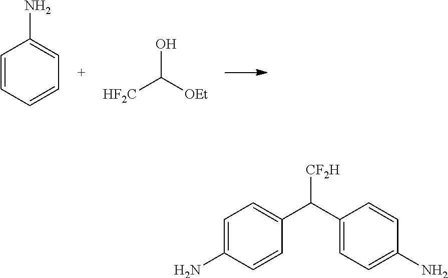

Aniline (0.8 mol, 2 equiv), difluoroacetaldehyde ethyl hemiacetal (50.4 g, 0.4 mol), hexafluoroisopropanol (640 mL), and triphenylborane (0.08 mol) were sequentially added into a 1,000 mL reaction flask to carry out a reaction at 50° C. According to monitoring by TLC plate spotting, the reaction was completed at 18 h. After completion of the reaction, the solvent was recovered by rotary evaporation, and a crude reaction product was recrystallized with ethanol to obtain 99.2 g of a product (4,4′-(2,2-difluoroethane-1,1-diyl)dianiline) of the above formula with a yield of 96%.

¹H NMR (400 MHz, CDCl₃) δ 7.07 (d, J=8.4 Hz, 4H), 6.67-6.62 (m, 4H), 6.19 (td, J=56.3, 4.4 Hz, 1H), 4.19 (td, J=16.2, 4.3 Hz, 1H), 3.63 (s, 4H); ¹⁹F NMR (376 MHz, CDCl₃) δ −118.04 (dd, J=56.6, 16.2 Hz, 2F); ¹³C NMR (100 MHz, CDCl₃) δ 145.5, 129.8, 127.5 (t, ³J_{C-F}=3.5 Hz), 117.3 (t, ¹J_{C-F}=244.9 Hz), 115.2, 53.4 (t, ²J_{C-F}=20.5 Hz); HRMS (ESI) m/z: [M+H]⁺ calculated value C₁₄H₁₅F₂N₂ 249.1198; measured value; 249.1195.

### Example 14

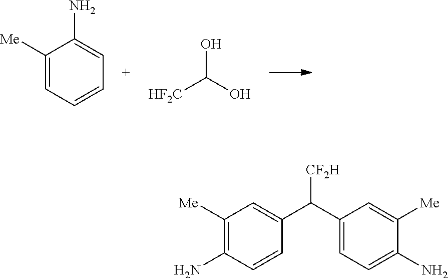

2-methylaniline (0.8 mol, 2 equiv), a difluoroacetaldehyde hydrate (39.2 g, 0.4 mol), hexafluoroisopropanol (640 mL), and tris(pentafluorophenyl)borane (0.04 mol) were sequentially added into a 1,000 mL reaction flask to carry out a reaction at 65° C. According to monitoring by TLC plate spotting, the reaction was completed at 12 h. After completion of the reaction, the solvent was recovered by rotary evaporation, and a crude reaction product was recrystallized with ethyl acetate to obtain 104.9 g of a product of the above formula with a yield of 95%.

### Example 15

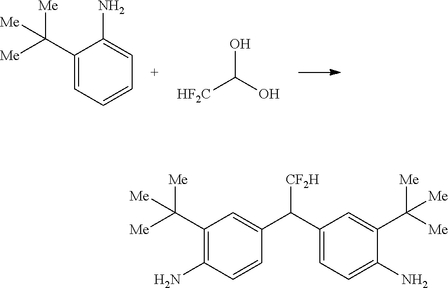

2-tert-butylaniline (0.8 mol, 2 equiv), a difluoroacetaldehyde hydrate (39.2 g, 0.4 mol), hexafluorobutanol (640 mL), and trimethylsilyl trifluoromethanesulfonate (0.04 mol) were sequentially added into a 1,000 mL reaction flask to carry out a reaction at 90° C. According to monitoring by TLC plate spotting, the reaction was completed at 14 h. After completion of the reaction, the solvent was recovered by rotary evaporation, and a crude reaction product was recrystallized with ethanol to obtain 138.2 g of a product of the above formula with a yield of 96%.

### Example 16

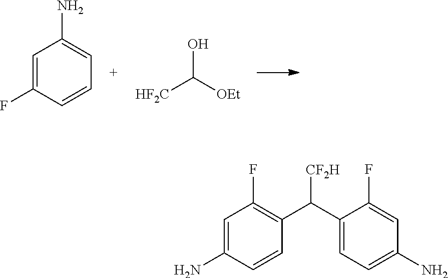

3-fluoroaniline (0.8 mol, 2 equiv), difluoroacetaldehyde ethyl hemiacetal (50.4 g, 0.4 mol), hexafluoroisopropanol (640 mL), and p-toluenesulfonic acid (0.04 mol) were sequentially added into a 1,000 mL reaction flask to carry out a reaction at 50° C. According to monitoring by TLC plate spotting, the reaction was completed at 14 h. After completion of the reaction, the solvent was recovered by rotary evaporation, and a crude reaction product was recrystallized with ethanol to obtain 77.2 g of a product of the above formula with a yield of 68%.

### Example 17

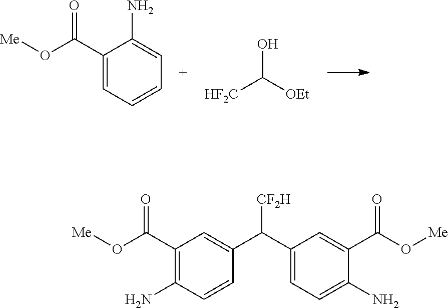

Methyl 2-aminobenzoate (0.96 mol, 2.4 equiv), difluoroacetaldehyde ethyl hemiacetal (50.4 g, 0.4 mol), trifluoroethanol (640 mL), and N-(trimethylsilyl)bis(trifluoromethanesulfonyl)imide (0.04 mol) were sequentially added into a 1,000 mL reaction flask to carry out a reaction at 65° C. According to monitoring by TLC plate spotting, the reaction was completed at 20 h. After completion of the reaction, the solvent was recovered by rotary evaporation, and a crude reaction product was recrystallized with ethanol to obtain 75.7 g of a product (dimethyl 5,5′-(2,2-difluoroethane-1,1-diyl)bis(2-aminobenzoate)) of the above formula with a yield of 52%.

¹H NMR (400 MHz, CDCl₃) δ 7.79 (d, J=2.2 Hz, 2H), 7.17 (dd, J=8.5, 2.2 Hz, 2H), 6.63 (d, J=8.5 Hz, 2H), 6.20 (td, J=56.0, 4.1 Hz, 1H), 5.70 (s, 4H), 4.17 (td, J=16.3, 4.0 Hz, 1H), 3.86 (s, 6H); ¹⁹F NMR (376 MHz, CDCl₃) δ −118.44 (dd, J=56.1, 16.3 Hz, 2F); ¹³C NMR (100 MHz, CDCl₃) δ 168.3, 149.6, 134.6, 131.4, 124.8 (t, ³J_{C-F}=3.4 Hz), 117.2, 116.9 (t, ¹J_{C-F}=245.2 Hz), 110.6, 53.0 (t, ²J_{C-F}=20.7 Hz), 51.6; HRMS (ESI) m/z: [M+H]⁺ calculated value C₁₈H₁₉F₂N₂O₄ 365.1307; measured value; 365.1305.

### Example 18

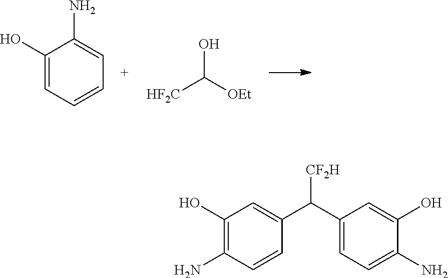

2-aminophenol (0.8 mol, 2 equiv), difluoroacetaldehyde ethyl hemiacetal (50.4 g, 0.4 mol), hexafluoroisopropanol (640 mL), and tris(2,6-difluorophenyl)borane (0.04 mol) were sequentially added into a 1,000 mL reaction flask to carry out a reaction at 65° C. According to monitoring by TLC plate spotting, the reaction was completed at 15 h. After completion of the reaction, the solvent was removed by rotary evaporation, and a crude reaction product was recrystallized with methanol to obtain 81.8 g of a product of the above formula with a yield of 73%.

### Example 19

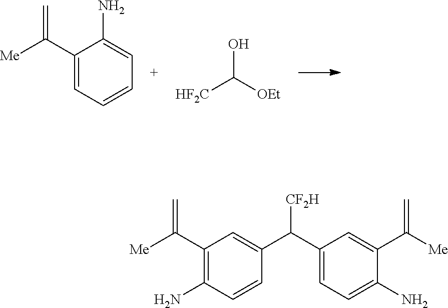

2-isopropenylphenylaniline (0.88 mol, 2.2 equiv), difluoroacetaldehyde ethyl hemiacetal (50.4 g, 0.4 mol), tetrafluorobutanediol (640 mL), and tris(2,5-bis(trifluoromethyl)phenyl)borane (0.04 mol) were sequentially added into a 1,000 mL reaction flask to carry out a reaction at 50° C. According to monitoring by TLC plate spotting, the reaction was completed at 18 h. After completion of the reaction, the solvent was recovered by rotary evaporation, and a crude reaction product was recrystallized with ethanol to obtain 66.9 g of a product of the above formula with a yield of 51%.

### Example 20

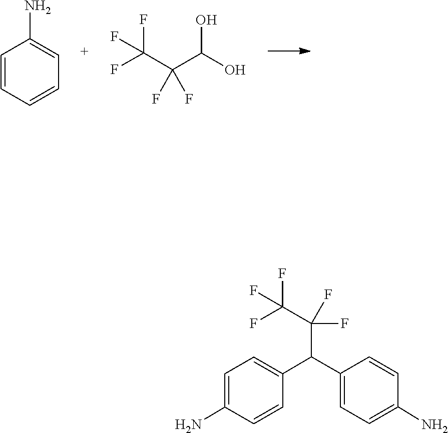 [SMILES: Nc1ccccc1 ; OC(O)C(F)(F)C(F)(F)F ; Nc1ccc(C(c2ccc(N)cc2)C(F)(F)C(F)(F)F)cc1]

Aniline (0.64 mol, 2 equiv), a pentafluoropropionaldehyde hydrate (53 g, 0.32 mol), hexafluoroisopropanol (640 mL), and tris(2,3,5,6-tetrafluoro-4-(trifluoromethyl)phenyl)borane (0.032 mol) were sequentially added into a 1,000 mL reaction flask to carry out a reaction at 65° C. According to monitoring by TLC plate spotting, the reaction was completed at 14 h. After completion of the reaction, the solvent was recovered by rotary evaporation, and a crude reaction product was recrystallized with ethanol to obtain 68.8 g of a product of the above formula with a yield of 68%.

Obviously, the above examples of the present invention are only exemplified for describing the present invention more clearly, and are not intended to limit the embodiments of the present invention. For those of ordinary skill in the art, other changes or modifications in different forms can also be made on the basis of the above description. It is impossible to enumerate all the embodiments, and any obvious changes or modifications derived from the technical solutions of the present invention still fall within the scope of protection of the present invention.

What is claimed is:

1. A method for synthesizing a fluoroalkyl-substituted 4,4′-diaminodiphenylmethane compound, comprising:
under the action of a catalyst, allowing an aromatic primary amine compound to react with a difluoroacetaldehyde hydrate, a trifluoroacetaldehyde a 2,2,3,3-tetrafluoropropionaldehyde hydrate, a pentafluoropropionaldehyde hydrate, a heptafluorobutyraldehyde hydrate, or a nonafluorovaleraldehyde hydrate in an organic solvent, and after completion of the reaction, performing post-treatment to obtain the fluoroalkyl-substituted 4,4′-diaminodiphenylmethane compound having a structure of general formula [1]: 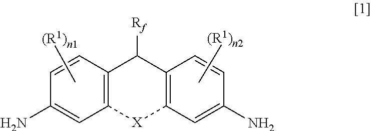
wherein in the general formula [1], n₁ and n₂ are respectively and independently an integer of 0-4, and in cases where multiple R¹ are present, respectively and independently represent a substituent;
R¹ is independently selected from hydrogen, alkyl, alkoxy, cycloalkyl, aryl, propenyl, halogen, hydroxyl, benzyl, and thioalkyl;
R_{f} is independently selected from difluoromethyl, trifluoromethyl, tetrafluoroethyl, pentafluoroethyl, heptafluoropropyl, and nonafluorobutyl;
X is independently selected from methylene, oxygen, or sulfur;
the catalyst is a Lewis acid or Brønsted acid;
the organic solvent is 2,2-difluoroethanol, hexafluoroisopropanol, hexafluoro-2-methylisopropanol, hexafluoro-2-phenylisopropanol, trifluoroethanol, perfluoroalkylethanol, perfluoro-tert-butanol, tetrafluoropropanol, tetrafluorobutanediol, hexafluorobutanol, or octafluoropentanol; and
the post-treatment comprises: after completion of the reaction, removing the solvent under reduced pressure, and recrystallizing a crude reaction product to obtain the fluoroalkyl-substituted 4,4′-diaminodiphenylmethane compound; and after completion of the reaction, recovering a fluoroalcohol solvent through a simple distillation operation.

2. The method for synthesizing a fluoroalkyl-substituted 4,4′-diaminodiphenylmethane compound according to claim 1, wherein R¹ is independently selected from hydrogen, methyl, ethyl, tert-butyl, isopropyl, alkoxy, cycloalkyl, phenyl, propenyl, halogen, hydroxyl, benzyl, and thioalkyl.

3. The method for synthesizing a fluoroalkyl-substituted 4,4′-diaminodiphenylmethane compound according to claim 1, wherein the Lewis acid is selected from phenylboronic acid, boric acid, trimethyl borate, triethyl borate, triisopropyl borate, tributyl borate, triphenyl borate, boron trifluoride diethyl etherate, tris(2,4-bis(trifluoromethyl)phenyl)borane, tris(pentafluorophenyl)borane, tris(2,2,2-trifluoroethyl)borate, triphenylborane, tris(hexafluoroisopropyl)borate, tris(2,3,5,6-tetrafluoro-4-(trifluoromethyl)phenyl)borane, tris(2,4-bis(trifluoromethyl)phenyl)borane, trimethylsilyl trifluoromethanesulfonate, tert-butyldimethylsilyl trifluoromethanesulfonate, trimethylsilyl acetate, trimethylsilylmethanesulfonate, N-(trimethylsilyl)bis(trifluoromethanesulfonyl)imide, (pentafluorophenyl)bis(trifluoromethanesulfonyl)trimethylsilylmethane, di-tert-butyl isobutylsilyl trifluoromethanesulfonate, tert-butyldiphenylsilyl trifluoromethanesulfonate, tert-butyldimethylsilyl trifluoromethanesulfonate, 2-(trimethylsilyl)phenyl trifluoromethanesulfonate, triisopropylsilyl trifluoromethanesulfonate, diisopropylsilyl bis(trifluoromethanesulfonate), diethylisopropylsilyl trifluoromethanesulfonate, di-tert-butylsilyl bis(trifluoromethanesulfonate), triethylsilyl trifluoromethanesulfonate, tris(2,6-difluorophenyl)borane, tris(2,5-bis(trifluoromethyl)phenyl)borane, 2,4,6-tris(3-(trifluoromethyl)phenyl)-1,3,5,2,4,6-trioxatriborinane, triphenylmethyl tetrakis(pentafluorophenyl)borate, bis(perfluorophenyl)borane, tris(2,4,6-trifluorophenyl)borane, tris[3,5-bis(trifluoromethyl)phenyl]borane, tris(2,2′,2″-perfluorobiphenyl)borane, bis(pentafluorophenyl)-(2-perfluorobiphenyl)borane, or tris(2-perfluoronaphthyl)borane; and
the Brønsted acid is selected from acetic acid, benzoic acid, benzenesulfonic acid, p-toluenesulfonic acid, methanesulfonic acid, camphorsulfonic acid, Amberlyst 15 ion exchange resin, diphenyl phosphate, binaphthol phosphate, trifluoroacetic acid, trifluoromethanesulfonic acid, lactic acid, oxalic acid, sulfuric acid, hydrochloric acid, perchloric acid, phosphoric acid, or polyphosphoric acid.

4. The method for synthesizing a fluoroalkyl-substituted 4,4′-diaminodiphenylmethane compound according to claim 1, wherein a reaction temperature is from room temperature to 130° C., and a reaction time is 12-24 h.

5. The method for synthesizing a fluoroalkyl-substituted 4,4′-diaminodiphenylmethane compound according to claim 2, wherein a reaction temperature is from room temperature to 130° C., and a reaction time is 12-24 h.

6. The method for synthesizing a fluoroalkyl-substituted 4,4′-diaminodiphenylmethane compound according to claim 3, wherein a reaction temperature is from room temperature to 130° C., and a reaction time is 12-24 h.

7. The method for synthesizing a fluoroalkyl-substituted 4,4′-diaminodiphenylmethane compound according to claim 1, wherein a molar ratio of the aromatic primary amine compound to the difluoroacetaldehyde hydrate, the trifluoroacetaldehyde hydrate, the 2,2,3,3-tetrafluoropropionaldehyde hydrate, the pentafluoropropionaldehyde hydrate, the heptafluorobutyraldehyde hydrate, or the nonafluorovaleraldehyde hydrate is (1-2.4): 1.

8. The method for synthesizing a fluoroalkyl-substituted 4,4′-diaminodiphenylmethane compound according to claim 1, wherein a molar ratio of the difluoroacetaldehyde hydrate, the trifluoroacetaldehyde hydrate, the 2,2,3,3-tetrafluoropropionaldehyde hydrate, the pentafluoropropionaldehyde hydrate, the heptafluorobutyraldehyde hydrate, or the nonafluorovaleraldehyde hydrate to the catalyst is 1:(0-0.2).

9. The method for synthesizing a fluoroalkyl-substituted 4,4′-diaminodiphenylmethane compound according to claim 7, wherein a molar ratio of the difluoroacetaldehyde hydrate, the trifluoroacetaldehyde hydrate, the 2,2,3,3-tetrafluoropropionaldehyde hydrate, the pentafluoropropionaldehyde hydrate, the heptafluorobutyraldehyde hydrate, or the nonafluorovaleraldehyde hydrate to the catalyst is 1:(0-0.2).
